(19) United States
(12) Patent Application Publication
(10) Pub. No.: US 2013/0131466 A1
(43) Pub. Date: May 23, 2013

(54) TOOL FOR SCREENING FOR A DEFECT, SUCH AS PERIPHERAL ARTERIAL DISEASE

(71) Applicants: Bruno Wacogne, Traitiefontaine (FR); Christian Pieralli, Besancon (FR); Stephane Roeslin, Roche Lez Beaupre (FR); Lionel Pazart, Besancon (FR); Christophe Roncato, La Rochelle (FR)

(72) Inventors: Bruno Wacogne, Traitiefontaine (FR); Christian Pieralli, Besancon (FR); Stephane Roeslin, Roche Lez Beaupre (FR); Lionel Pazart, Besancon (FR); Christophe Roncato, La Rochelle (FR)

(73) Assignees: CENTRE HOSPITALIER UNIVERSITAIRE DE BESANCON, BESANCON CEDEX (FR); UNIVERSITE DE FRANCHE-COMTE, BESANCON CEDEX (FR)

(21) Appl. No.: 13/813,220

(22) PCT Filed: Jul. 28, 2011
(86) PCT No.: PCT/FR11/51831

§ 371 Date: Jan. 30, 2013

(30) Foreign Application Priority Data
Jul. 30, 2010 (FR) 1056318

(51) Int. Cl. A61B 5/021; A61B 5/1455

(52) U.S. Cl. CPC A61B 5/02141; A61B 5/1455
USPC 600/301

(57) ABSTRACT

A screening tool includes: a compression body for engaging with a portion of a limb of the patient to be compressed; a compression element for compressing the contact surface against the portion of the patient's limb to be compressed; a measurement element for structurally and functionally engaging with a portion of the limb to be examined, as well as generating, via the optical detection of one or more component(s) of the blood, a measurement signal that is characteristic of an arterial pulse in the portion of the limb to be examined during a measurement phase. The compression element generates a measurement pressure substantially equal to a threshold pressure value during the entire measurement phase, and the screening tool also includes: a calculation element for transmitting a negative signal when the arterial pulse is greater than a threshold diagnostic value, and a positive signal it is lower than the threshold value.

The invention relates generally to the field of screening patients for peripheral arterial disease of the limbs, in particular of the lower limbs.

Peripheral arterial disease of the lower limbs is a common complication in diabetes since it affects between 17 and 21% of the diabetic population. This vascular disease, associated with a minor trauma and with diabetic neuropathy, is the cause of ulceration of the diabetic foot, which precedes approximately 85% of amputations. Simple measures can be taken to prevent this dreadful complication when this vascular disease is indentified in time.

In this regard, the invention relates to a tool comprising a compression body having a contact surface, the compression body being capable of engaging with a portion to be compressed of a limb of the patient, such that the contact surface comes into non-punctiform or virtually non-punctiform contact with the portion to be compressed of the limb of the patient; compression means capable of compressing this contact surface against this portion to be compressed of the limb of the patient, in such a way as to generate, on said portion to be compressed, a measurement pressure that is substantially homogeneous and equal to a pressure threshold value during a compression phase; measurement means capable, firstly, of engaging structurally and functionally with a portion to be examined of the limb of the patient and, secondly, of generating, by optical detection of one or more components of the blood, a measurement signal that is characteristic of an arterial pulse in the portion to be examined of the limb of the patient during a measurement phase.

An analogous device is known to a person skilled in the art, in particular from the example thereof given in the document U.S. Pat. No. 5,050,613, which describes a proven method consisting in measuring the systolic pressure index, hereinafter the SPI, of the ankle of the patient using a capillary vascular testing appliance that comprises inflatable bands and also blood flow sensors and blood pressure sensors, and allowing collection of data concerning the blood flow in the ankle of the patient. More precisely, this appliance is used first of all by inflating the inflatable bands until the arteries of the patient are occluded, and, subsequently, by then deflating these inflatable bands until the arteries re-open. It is then necessary to carry out a measurement not only when the arteries of the patient are occluded, but also during the phase of deflation of the bands, when said arteries re-open.

However, the disadvantage of this measurement of the SPI is that it is easily disrupted in diabetic patients, on account of the frequent presence of calcifications in the wall of the artery. Consequently, the results are not sufficiently reliable for the population to whom this method is addressed.

In order to overcome this major disadvantage, the prior art discloses so-called microcirculatory techniques as alternatives to measuring the SPI of the ankle.

The first of these techniques is employed by an appliance marketed under the trademarks Perimed® and Periflux System 5000®. This appliance comprises a compression body and compression means, which are intended to apply different compression pressures to a portion to be compressed of the patient's big toe, and a system for compression of the arterial flow, based on laser Doppler technology, for measuring the arterial pressure in the big toe. It can also be equipped with a probe for measuring the transcutaneous oxygen pressure (TcPo2). The analysis of the results thereby obtained allows practitioners to make a relatively reliable diagnosis of the hemodynamic state of the lower limbs of the patient and, therefore, of the peripheral arterial disease of the lower limbs of the patient.

However, this first technique also has disadvantages. Firstly, the examinations carried out to obtain the measurements necessary for establishing these diagnoses require lengthy examination protocols, of which the time-consuming nature is a serious obstacle to the frequent and regular use of this technique. Secondly, the cost of investment in such an appliance is often prohibitive and therefore limits its use to a few specialized hospital centers. Thirdly, establishing a reliable diagnosis necessarily entails the involvement of a specialized practitioner who is able to analyze the curves showing a signal intensity representative of the arterial flow rate as a function of the different compression pressures. Now, it is well known that the availability of these specialized practitioners is not sufficient to allow these techniques to be widely used.

The other technique providing an alternative to the measurement of the SPI concerns an appliance marketed by the company Atys Medical® under the trademark Systoe®. In the same way as above, this appliance comprises a compression body and compression means that are intended to apply different compression pressures to the portion of the patient's big toe that is to be compressed. However, unlike the previous appliance, this one uses the principle of photoplethysmography, which consists in using, as the detection principle, the relationship between the intensity of the light reflection on the portion to be examined and the blood flow passing through the zone being studied, according to the variation in compression pressure imposed on the portion to be compressed.

Although the principle of photoplethysmography makes this technique more compact and transportable, it is no less problematic in several respects. Firstly, relating the compression pressures with the measurements obtained by photoplethysmography involves the use of equipment of which the costs remain prohibitive for certain non-specialized medical facilities, for general practitioners, and for the patients themselves. In addition, the analysis of the measurements thereby obtained remains long and overly complex, and therefore a person not specialized in the field in question is unable to make a reliable diagnosis.

In addition, the prior art also includes the document WO-A1-2004/073514, which describes a diagnostic device comprising a compression system capable of exerting a predetermined pressure on the surface of a tissue in order to initiate blanching therein, and then of releasing this pressure after a predetermined time. In parallel, the device incorporates an optical system which illuminates the tissue and collects data on reflected light, during the blanching phase and after the blanching phase, so as to allow a micro-controller to establish a diagnosis of the tissues in question. According to this document WO-A1-2004/073514, it is thus necessary to carry out a measurement not only during the blanching phase, but also during the recovery phase that follows it. This is because the micro-controller is not capable of emitting a diagnosis on the basis of a signal measured by the optical system during the compression phase alone.

As before, this solution entails relating the pressure measurements with the data on the reflected light obtained, and using an algorithm for complex classification of the results, which makes it necessary to use equipment that is even more expensive and, consequently, prohibitive for non-specialists.

A measurement device intended to diagnose the state of a patient's limbs is likewise known from the document U.S. Pat. No. 5,755,229. This device comprises an inflatable band that can be wound around the limb of the patient, a pump, an air conduit and a pressure sensor that are capable of inflating the inflatable band to several predefined pressures, and an optical-signal sensor permitting measurement of the plethysmogram of the limb in question. The device additionally comprises a CPU for establishing a diagnosis on the basis of a plurality of signals corresponding to the pulses measured by the optical-signal sensor when different pressures are applied to the limb of the patient.

The disadvantage of such a solution is that it once again requires obtaining optical measurements performed on the basis of different pressure values applied to the limb of the patient, and relating these optical measurements to the pressure values applied. Such a solution is therefore complex in that it requires simultaneous processing of the pressure means and of the measurement means.

There are therefore requirements that have not been resolved by the prior art.

In this context, the object of the present invention is to make available a device that is free of at least one of the abovementioned limitations.

More particularly, the invention aims to make available a screening tool which screens for a defect such as peripheral arterial disease and which is both simple to use and also less costly than the existing devices.

To this end, the screening tool according to the invention, and also in accordance with the generic definition thereof given in the preamble above, is principally characterized in that it comprises, firstly, calculation means which, on the basis of a measured signal corresponding to the measurement signal generated by the measurement means during the compression phase, are capable of emitting a negative diagnostic signal when the arterial pulse in the portion to be examined is higher than a diagnostic threshold value, and of emitting a positive diagnostic signal when this arterial pulse is lower than this diagnostic threshold value, and, secondly, expression means capable of expressing negative diagnostic information upon receipt of the negative diagnostic signal from the calculation means.

By virtue of this configuration, it is possible to screen for a defect, such as peripheral arterial disease, without having to use a compression technique involving variable compression of the arteries. Moreover, the compression means and the measurement means can be treated completely independently, since it is no longer a question of correlating a pressure measurement with the signal measured by the measurement means. On the contrary, in this case, the compression phase, during which the measurement pressure is applied to the portion to be examined, is maintained throughout the duration of the measurement phase. Thus, it is no longer necessary to use a control system between these two subassemblies, which greatly simplifies the device and makes it possible, firstly, to improve the compact nature of the device and its portability and, secondly, to greatly reduce the costs of production of the appliance. In the same way, this arrangement makes it unnecessary to analyze several measurement curves in order to interpret them in relation to one another. For this reason, it is possible to express diagnostic information without the need for involvement of practitioners specialized in this field. It is thus possible to imagine screening tests being carried out by the patients themselves, if appropriate supplemented by complementary examinations in the event of positive diagnosis. A fortiori, this screening tool, which does not require any medical interpretation after the measurements have been carried out, is also much quicker to use and therefore saves considerable time in carrying out the screening.

According to one embodiment, the compression body comprises at least one flexible portion made of a flexible and atraumatic material. This allows the compression body to be held in position in a way that is both reliable and also comfortable for the patient.

In a first possible embodiment, the compression body comprises the at least one flexible portion and a rigid or semi-rigid peripheral wall; the at least one flexible portion is permanently fixed, directly or indirectly, on the peripheral wall; and the contact surface is formed partly or wholly on the at least one flexible portion.

In this case, provision can be made that the at least one flexible portion comprises an internal chamber capable of containing, in a hermetic manner, a fluid introduced into this internal chamber by way of a fluid inlet.

In this case, provision can likewise be made that the compression body comprises a single flexible portion made of a flexible and atraumatic material, and a peripheral wall; that the single flexible portion is permanently fixed, directly or indirectly, on the peripheral wall; and that the contact surface is formed entirely on the single flexible portion.

In a second possible embodiment, the compression body comprises a sleeve, with: an outer and peripheral wall made of a flexible and atraumatic material; a fluid inlet corresponding to a structural and functional opening made in the outer and peripheral wall; and an internal chamber delimited by the outer and peripheral wall and capable of containing, in a hermetic manner, a fluid introduced into this internal chamber by way of the fluid inlet. This second embodiment makes it possible to obtain a diagnostic tool that is particularly simple, both in terms of its production and its use.

According to one embodiment for which the compression body comprises an internal chamber, the compression means comprise a delivery member capable of generating a flow of fluid, and transfer means capable of transporting the fluid from the delivery member as far as the fluid inlet of the compression body, in such a way as to transfer the fluid from the compression means to the internal chamber of the compression body via this fluid inlet. Thus, the portion to be compressed can be compressed in a reliable, simple and inexpensive manner.

In this case, the delivery member of the compression means can comprise a bellows, a pump or the like.

In one embodiment, the compression means comprise a calibrated valve capable of allowing some of the fluid to escape from the internal chamber of the compression body as long as said fluid generates, in this compression chamber, a measurement pressure higher than a predetermined pressure threshold value. This embodiment makes it possible to obtain, in a particularly reliable manner, a measurement pressure equal to the pressure threshold value, specifically throughout the compression phase.

In this case, it is likewise possible that the calibrated valve is capable of changing from a first state, in which the pressure threshold value corresponds to a first predetermined threshold value, to a second state, in which the pressure threshold value corresponds to a second predetermined threshold value. The patient can thus carry out two different screening tests using two distinct measurement pressures.

According to one embodiment, the flexible material is a silicone.

In a third possible embodiment, the compression body comprises an inextensible band capable of being deformed in such a way as to fit tightly on the portion to be compressed of the limb of the patient; the band having a width of greater than 2 mm (millimeters).

In this case, the compression means are capable of exerting a tightening force on the inextensible band of the compression body when this inextensible band fits tightly on the portion to be compressed, in such a way as to generate, on said portion to be compressed, a measurement pressure substantially equal to a pressure threshold value throughout the compression phase.

In this case too, the compression means comprise an elastic element fixed permanently on one end of the inextensible band; the elastic element being capable of changing from a relaxed position to a tensioned position in which said elastic element generates a return force corresponding to the tightening force.

According to particular embodiments:
the compression body is a disposable element, by which means it is possible to avoid bacterial transmission between different patients;
the compression means are capable of generating, on the portion to be compressed, a fixed or non-fixed measurement pressure situated in a range of 30 to 80 mmHg (millimeters of mercury), in which range the measurements allow significant results to be obtained.

According to one embodiment, the measurement means comprise a light source, such as a laser diode, capable of emitting rays of light toward the portion to be examined of the limb of the patient, the emitted rays of light having predetermined emission characteristics; a receiver, such as a photodiode, capable of receiving the rays of light transmitted or backscattered by the portion to be examined of the limb of the patient, the received rays of light having predetermined reception characteristics; and a calculation unit capable of comparing the emission characteristics with the reception characteristics, in such a way as to deduce therefrom the measurement signals that are characteristic of the arterial pulse in the portion to be examined of the limb of the patient. Such measurement means have the advantage of being both compact and transportable.

According to one embodiment, the calculation means are capable of deducing, from the measurement signal, a useful signal that is characteristic of the arterial pulse in the portion to be examined of the limb of the patient.

In this case, when the measurement signal is composed of a pseudo-continuous spurious component and of a useful component having a measurement tension that is characteristic of the arterial pulse, the calculation means are capable of eliminating the pseudo-continuous spurious component of the measurement signals and of amplifying the measurement tension of the useful component.

In this case too, the calculation means are capable of comparing the amplified measurement tension with a comparison threshold tension, in such a way as to emit a negative diagnostic signal when the amplitude of the amplified measurement tension is higher than the amplitude of the comparison threshold tension, and a positive diagnostic signal when the amplitude of the amplified measurement tension is lower than the comparison threshold tension.

In this case again, either the light source and the receiver are capable of being positioned on either side of the portion to be examined of the limb of the patient, such that the received rays of light correspond to the emitted rays of light having passed through the portion to be examined of the limb of the patient, or the light source is capable of being positioned in proximity, such that the received rays of light correspond to the emitted rays of light backscattered by the portion to be examined of the limb of the patient.

In the case where the received rays of light correspond to the emitted rays of light backscattered by the portion to be examined, the light source can be linked structurally to the receiver; the light source and the receiver being arranged together in a receiving element.

According to one embodiment for which the compression body comprises a sleeve, the light source and the receiver are positioned in the internal chamber delimited by the outer wall of the sleeve.

According to one embodiment for which the compression body comprises a rigid or semi-rigid peripheral wall, the measurement means comprise a receiving element having a protective outer wall delimiting a protected internal space, the light source being positioned at least partially inside the protected internal space.

In this case, the receiver can likewise be positioned at least partially inside the protected internal space.

In this case too, the receiving element may be capable of being placed and held in position, directly or indirectly, by the rigid or semi-rigid peripheral wall of the compression body.

In the preceding case, the receiving element can then be capable of being placed and held in position, indirectly, by the rigid or semi-rigid peripheral wall; the receiving element being arranged between two flexible portions.

According to one embodiment, the screening tool likewise comprises heating means that are capable of maintaining or increasing the temperature of at least one portion of the limb of the patient.

In this case, the heating means may be capable of maintaining or increasing the temperature of the portion to be compressed and/or the portion to be examined of the limb of the patient.

In this case too, the heating means can comprise an item of clothing that is capable of covering and enveloping at least one portion to be covered of the limb of the patient; the item of clothing being formed from a thermally insulating fiber, in such a way as to maintain or increase the temperature of the at least one portion to be covered of the limb of the patient.

In this case too, the diagnostic tool can comprise a temperature probe capable of measuring the temperature of at least one portion of the limb of the patient, preferably the portion to be examined of the limb of the patient.

More precisely, the temperature probe can be integrated in the item of clothing.

In the case where the heating means comprise an item of clothing, the item of clothing can incorporate the compression body, the compression means and/or the measurement means.

In the case where the heating means comprise an item of clothing, the item of clothing can be disposable.

According to one embodiment, the limb of the patient is a foot, the portion to be compressed and the portion to be examined being positioned on the big toe.

According to one embodiment, the portion to be compressed is positioned on the first phalanx of the big toe, and the portion to be examined is positioned on the second phalanx of the big toe.

According to one embodiment, the portion to be compressed and the portion to be examined are positioned on the second phalanx of the big toe.

According to one embodiment, the item of clothing is capable of insulating the big toe from the other toes of the patient.

Other features and advantages of the invention will become clear from the following description which is given by way of example and is non-limiting and in which reference is made to the attached drawings, where:

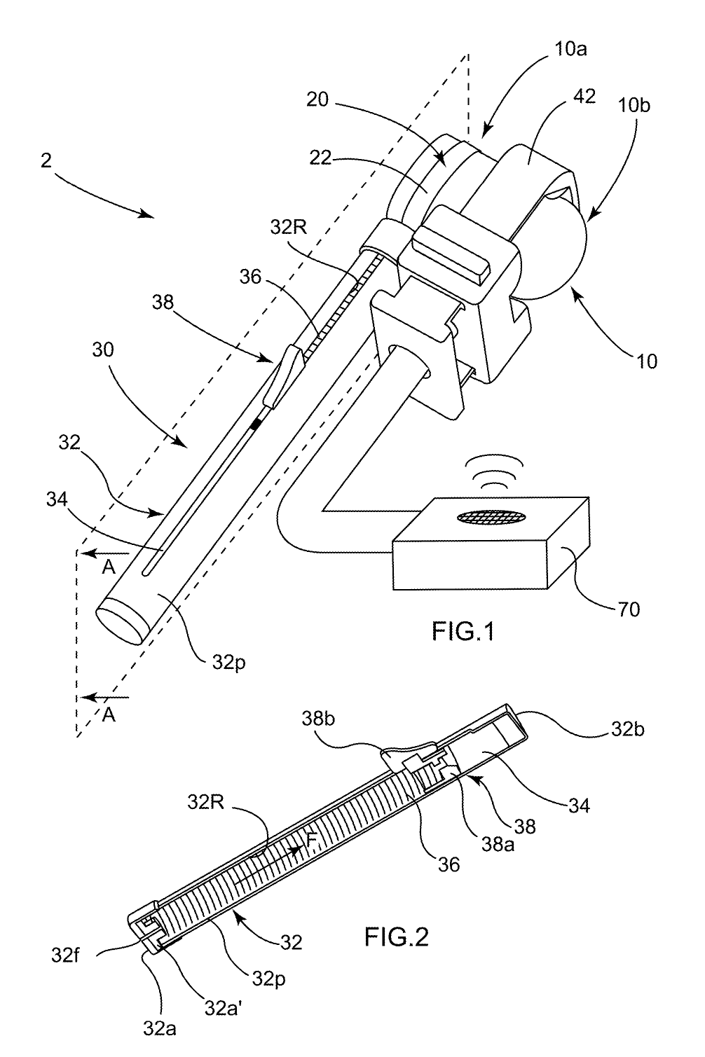

FIG. 1 is a perspective view of a first embodiment of the screening tool according to the invention, in which the compression body and the compression means are structurally independent of the measurement means;

FIG. 2 is a sectional view A-A of the compression means of the screening tool illustrated by FIG. 1;

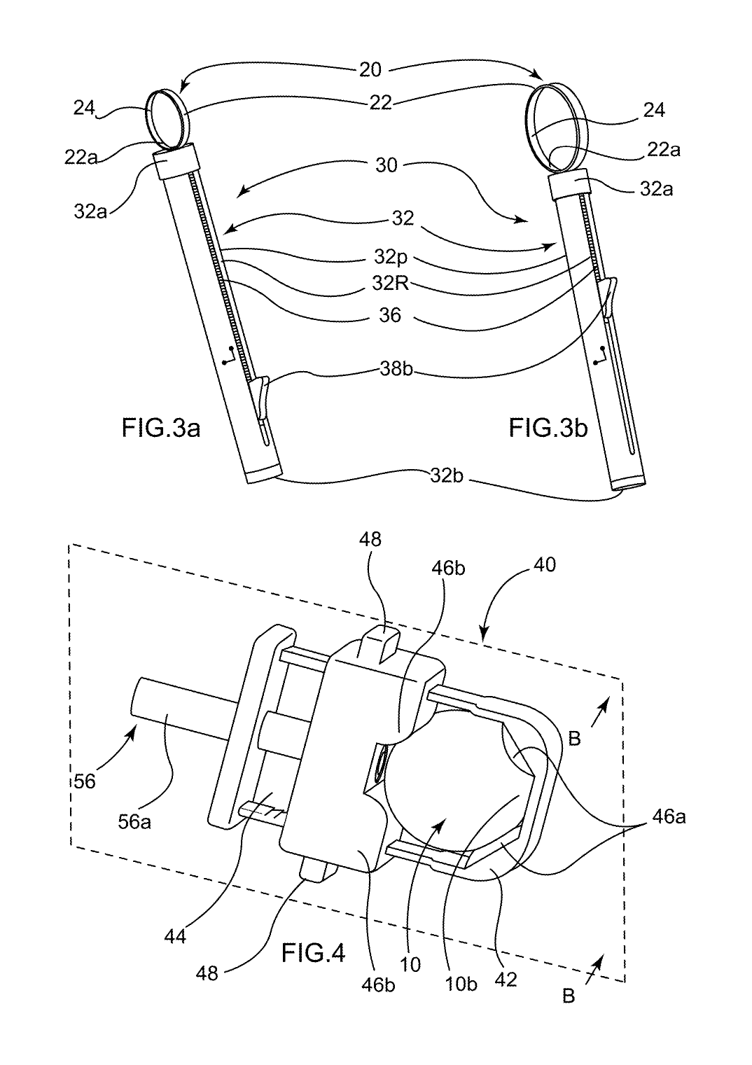

FIG. 3a is a perspective view of the compression body and of the compression means of the screening tool illustrated by FIG. 1, placed in a tensioned position;

FIG. 3b is a perspective view of the compression body and of the compression means of the screening tool illustrated by FIG. 1, which are placed in a relaxed position;

FIG. 4 is a perspective view of the measurement means of the screening tool illustrated by FIG. 1;

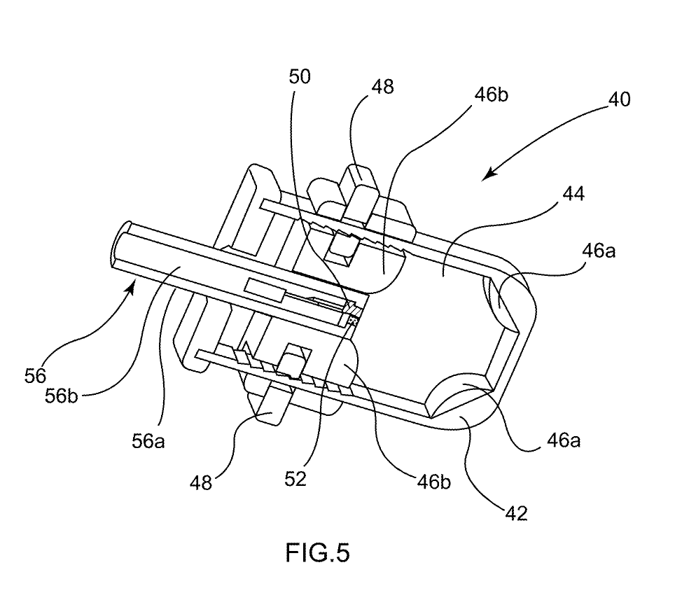

FIG. 5 is a sectional view B-B of the measurement means of the screening tool according to the invention illustrated by FIG. 1;

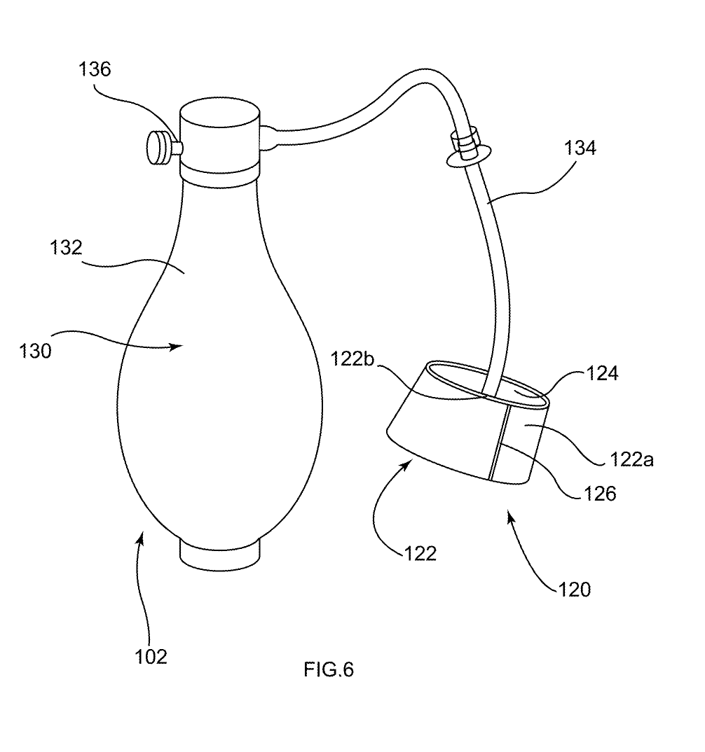

FIG. 6 is a perspective view of a second embodiment of the screening tool according to the invention, in which the compression body comprises an inflatable sleeve;

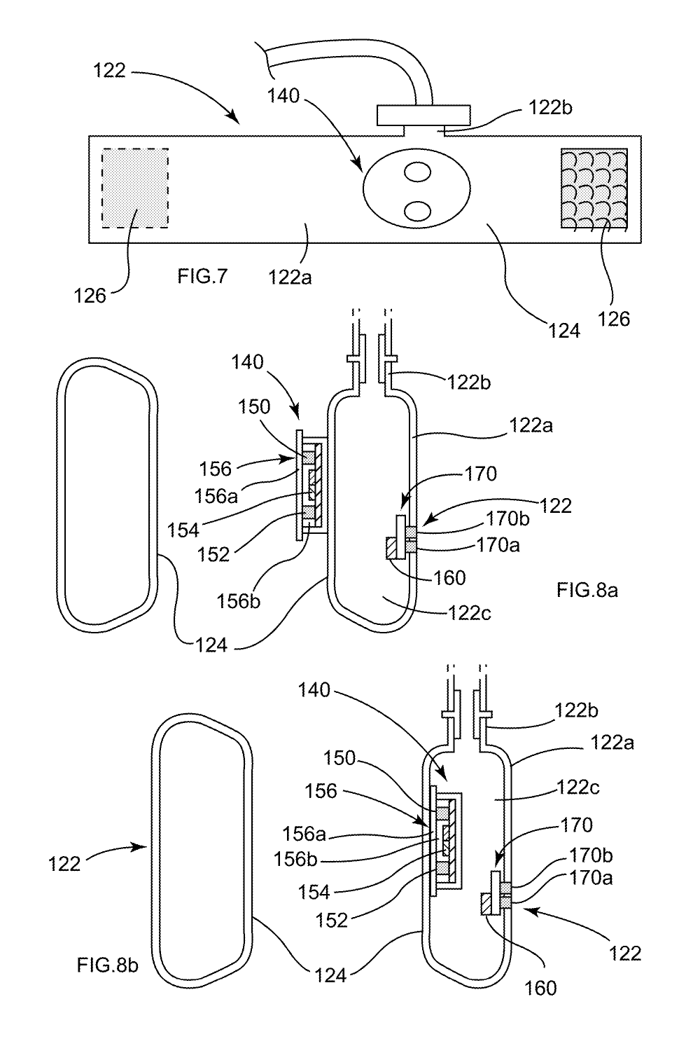

FIG. 7 is a schematic representation of the screening tool illustrated by FIG. 6, in a tensioned position;

FIG. 8a is a schematic representation, in section, of the screening tool illustrated by FIG. 6, in a tensioned position, and in which the measurement means comprise a light source and a receiver that are arranged outside the inflatable sleeve;

FIG. 8b is a schematic representation, in section, of the screening tool illustrated by FIG. 6, in a tensioned position, and in which the measurement means comprise a light source and a receiver that are arranged inside the inflatable sleeve;

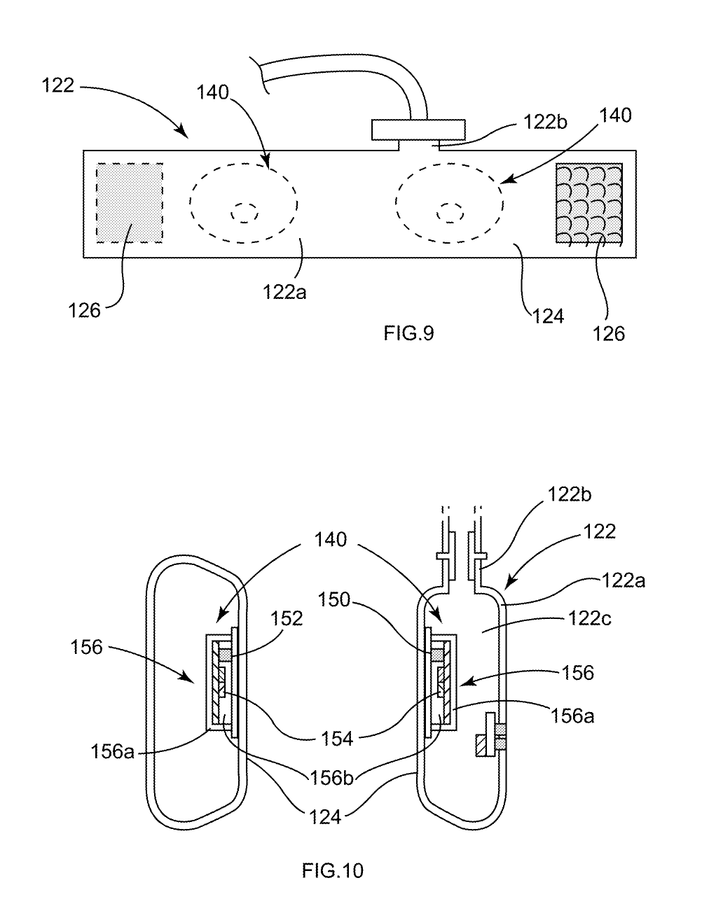

FIG. 9 is a perspective view of a third embodiment of the screening tool according to the invention, in which the compression body comprises an inflatable sleeve, and the measurement means function in transmission;

FIG. 10 is a schematic representation, in section, of the screening tool illustrated by FIG. 9 in a tensioned position.

It should first be noted that the screening tool 2 according to the invention is used particularly in the field of screening for a defect such as peripheral arterial disease of the lower limbs 10.

However, this defect is cited only by way of example and is non-limiting, and it must therefore be clearly appreciated that the invention likewise applies to screening for any other defect for which it would be useful, firstly, to compress a portion to be compressed 10a and, secondly, to carry out measurements by optical detection of one or more components of the blood on a portion to be examined 10b.

These screening operations can be performed on the big toe, as will principally be the case in the description below. However, they can likewise be performed on other limbs 10 of a patient, such as the wrist, the ankle, a finger, a toe other than the big toe, or even a stump, depending on the defect to be diagnosed and the corresponding mode of operation.

For practical reasons, reference will be made in the text below to screening for peripheral arterial disease of the lower limbs 10 only, the limb 10 to be considered being the big toe of the patient.

The embodiment in FIG. 1 will now be described in detail.

As has been indicated above, FIG. 1 illustrates a first embodiment of a screening tool 2 according to the invention intended to identify a peripheral arterial disease in the lower limbs of a patient.

This screening tool 2 comprises a compression body 20 formed by an inextensible band 22 with a first end 22a, a second end 22b and an inner face, called the contact surface 24, which is intended to bear on the portion to be compressed 10a of the limb 10 of the patient. The inextensible band 22 is likewise able to deform in such a way as to tightly fit the portion to be compressed 10a of the limb 10 of the patient. This inextensible band 22 has a length sufficient to surround the portion to be compressed 10a of the limb 10 of the patient, and a width greater than 2 millimeters, preferably equal to 15 millimeters.

In this way, when the inextensible band 22 of the compression body 20 engages with the portion to be compressed 10a, the contact surface 24 comes into non-punctiform or virtually non-punctiform contact with this portion to be compressed 10a. More particularly, the width of the inextensible band 24, and the fact that the contact is neither punctiform nor virtually punctiform, ensures that the portion to be compressed 10a does not sustain trauma, and instead it is possible to compress it in a substantially homogeneous manner, without causing the patient discomfort.

According to one embodiment, the inextensible band used can be disposable and can be replaced for each new screening operation. Thus, the risks of bacterial contamination between patients are limited.

The dimensions of this inextensible band 22 are such that it is able to fit tightly on the portion to be compressed 10a of the limb 10 of the patient. In this example, this portion to be compressed 10a corresponds to the first phalanx of the big toe, but if the portion to be compressed 10a was, for example, an ankle, the length of the inextensible band 22 would be made greater.

The screening tool in FIG. 1 likewise comprises compression means 30 capable of compressing the contact surface 24 of the inextensible band 22 against the portion to be compressed 10a. These compression means 30 comprise a support tube 32, inside which is defined a receiving cavity 34 in which an elastic element 36, such as a spring, is arranged. All three of these parts, namely the support tube 32, the receiving cavity 34 and the elastic element 36, extend in a longitudinal direction. Moreover, these compression means 30 likewise comprise a slide element 38.

FIGS. 2, 3a and 3b illustrate in greater detail the combination of the compression body 20 and the compression means 30.

As is shown in FIG. 2, the support tube 32 comprises a peripheral wall 32p, a first end 32a, a second end 32b, a guide groove 32r formed on the peripheral wall 32p, and a slit 32f formed on the first end 32a of the support tube 32. The guide groove 32r, which likewise extends in the longitudinal direction, defines, like the slit 32f, an opening toward the receiving cavity 34.

The slide element 38 has an inner portion 38a arranged inside the receiving cavity 34 and capable of sliding in this receiving cavity 34 in the longitudinal direction. The slide element 38 likewise comprises an outer portion 38b outside the receiving cavity 34 and extending through the guide groove 32r. The elastic element 36 is positioned between the first end 32a and the inner portion 38a of the slide element 38. In this way, when the outer portion 38b of the slide element 38 is moved toward the first end 32a of the support tube 32, the elastic element 36 is compressed by the inner portion 38a of the slide element 38. This elastic element 36 then generates a return force F tending to move the slide element 38 toward the second end 32b of the support tube 32.

It will be appreciated, in light of FIG. 2, that the first end 22a of the inextensible band 22 is permanently fixed structurally to the first end 32a of the support tube 32, more precisely between the first end 32a of the support tube 32 and a slotted ring 32a′. The inextensible band 22 forms a loop outside the support tube 32, passes through the slit 32f, the slotted ring 32a′ and the elastic element 36, and its second end 22b is fixed in the inner portion 38a of the slide element 38.

Thus, as is illustrated in FIG. 3a, in order to use the compression means 30, the outer portion 38b of the slide element 38 is moved toward the first end 32a of the support tube 32 in such a way as to compress the elastic element 36. Thus, the second end 22b of the inextensible band 22 moves toward the first end 32a of the support tube 32 and relaxes this inextensible band 22. The lasso formed by the inextensible band 22 can then be engaged around the portion to be compressed 10a of the limb 10 of the patient.

By convention, this position is called the relaxed position, since the inextensible band 22 is relaxed.

By contrast, as is illustrated in FIG. 3b, when the user releases the slide element 38, the elastic element 36 relaxes, generates a return force F corresponding to a tightening force and pushes the slide element 38 back toward the second end 32b of the support tube 32. Thus, the second end 22b of the inextensible band 22 likewise moves toward the second end 32b of the support tube 32, and the lasso formed by the inextensible band 22 then tightens around the portion to be compressed 10a. The inextensible band 22 is then tensioned around the portion to be compressed 10a of the limb 10 of the patient.

By convention, this position is called the tensioned position, since the inextensible band 22 is tensioned.

With this configuration, the pressure exerted by this inextensible band 22 on the portion to be compressed 10a can be adjusted as a function of the geometry of the compression body 20 and of the compression means 30 used. To do this, the stiffness of the elastic element must be calculated using the customary formula linking the force to the pressure:

P=F/S=constant=pressure threshold value, with
F: the force exerted by the inextensible band 22 on the portion to be compressed 10a (expressed in Newton), which is perpendicular to the contact surface 24 and which is uniformly distributed over the whole of this contact surface 24;
S: the surface area between the portion to be compressed 10a and the contact surface 24 of the inextensible band 22.

These compression means 30 are thus able to exert a tightening force on the inextensible band 22 of the compression body 20 such that, when this inextensible band 22 tightly fits on the portion to be compressed 10a, it generates, on said portion to be compressed 10a, a measurement pressure substantially equal to a previously determined pressure threshold value, and does this during a phase called the compression phase.

According to the embodiment used in screening for peripheral arterial disease of the lower limbs by a measurement performed on the big toe, this pressure threshold value is between 35 and 40 mmHg (millimeters of mercury), at which values an absence of arterial circulation could be attributable to a defect.

It should also be emphasized that, according to this embodiment, the elastic element 36 acts by compression on the slide element 38. However, an alternative embodiment is conceivable in which the elastic element 36 would be positioned between the second end 32b of the support tube 32 and the slide element 38, and in which the elastic element 36 would then act by traction on the slide element 38.

FIGS. 4 and 5 will now be described in detail.

FIGS. 4 and 5 illustrate the measurement means 40 used by the screening tool, illustrated in FIG. 1, during a measurement phase carried out after the compression body 20 has been fitted in place and the compression means 30 have been actuated.

These measurement means 40 are capable of engaging structurally and functionally with the portion to be examined 10b of the limb 10 of the patient, that is to say, in this embodiment, with the second phalanx of the big toe. These measurement means 40 comprise a peripheral frame 42 of rectangular shape, defining a receiving space 44 and supporting two flexible abutment portions 46a, which are arranged at two adjacent corners of the peripheral frame 42, and two flexible supporting portions 46b, which are arranged inside the receiving space 44.

The flexible abutment portions 46a and supporting portions 46b are made of silicone, for example.

Advantageously, the two flexible supporting portions 46b are fixed structurally and functionally with respect to each other but are capable of moving with respect to the peripheral frame 42. For this purpose, clamping pieces 48 allow the flexible supporting portions 46b to be blocked with respect to the peripheral frame 42 when a suitable position is found. This suitable position corresponds to a position in which the portion to be examined 10b of the limb 10 of the patient is held tightly by the two flexible supporting portions 46b and by the two flexible abutment portions 46a.

It should be emphasized that the rectangular shape of the peripheral frame 42 means that the portion to be examined 10b and the measurement elements can be received while at the same time allowing clamping of this portion to be examined 10b, without any great loss of space. However, it would also be possible to use other parallelepipedal, cylindrical and oval shapes or the like.

The measurement means 40 likewise comprise a light source 50, such as a laser diode, a receiver 52, such as a photodiode, and a calculation unit 54. The light source 50 is capable of emitting rays of light toward the portion to be examined of the limb of the patient, these emitted rays of light having predetermined emission characteristics. The receiver 52, for its part, is capable of receiving the rays of light backscattered by the portion to be examined of the limb of the patient, these received rays of light likewise having predetermined reception characteristics.

Such measurement means 40 thus make it possible to carry out a backscatter measurement during a phase called the measurement phase.

Alternatively, however, the light source 50 and the receiver 52 can be positioned on either side of the portion to be examined 10a. The receiver 52 is then situated opposite the light source 50 and integrated on the opposite portion of the peripheral frame 42. In this case, the received rays of light correspond to the emitted rays of light having passed through the portion to be examined 10b of the limb 10 of the patient, which corresponds to a measurement by transmission.

According to the embodiment in FIGS. 4 and 5, the measurement means 40 likewise comprise a receiving element 56, in this case a tube having an outer protective wall 56a that delimits a protected inner space 56b. The light source 50 and the receiver 52 are positioned at least partly inside the protected inner space. Moreover, this receiving element 56 is arranged between, and moves with, the two flexible supporting portions 46b, which hold it in position.

According to this embodiment, the calculation unit 54, which is connected electrically to the light source 50 and to the receiver 52, can be situated inside the protected inner space 56b too, or else in a separate protective housing. This calculation unit 54 makes it possible to compare the emission characteristics of the emitted rays of light with the reception characteristics of the received rays of light. Thus, this calculation unit 54 can generate, by optical detection of one or more components of the blood, a measured signal corresponding to the measurement signal generated by the measurement means 40 during the compression phase. The measured signal is thus characteristic of the arterial pulse in the portion to be examined 10b when the latter is subjected to the measurement pressure. This embodiment is advantageous since, given that the measured signal is generated during the compression phase, it is not necessary to correlate it with the development of the pressure.

It should be emphasized that, according to the embodiment in FIG. 1, the compression body 20 and the compression means 30 are structurally independent of the measurement means 40. Indeed, there is nothing structurally connecting this compression body 20 and these compression means 30 to the measurement means 40. To use the screening tool 2, it is therefore necessary, firstly, to compress the portion to be compressed 10a by way of the compression body 20 and the compression means 30, then to activate the measurement means 40 and hold the compression means 30 for a sufficient length of time such that the signal measured during this compression phase allows the calculation means 60 to generate a diagnostic signal. A preliminary measurement can additionally be carried out, before compression, in order to ascertain that the screening tool functions properly. However, according to an alternative embodiment, provision can also be made to connect the compression means 30 to the measurement means 40 so as to transmit an activation signal to these measurement means 40 when the pressure threshold value is reached. Thus, the measurement means 40 can automatically activate as soon as, and for as long as, the portion to be compressed 10a is compressed as it should be.

The screening tool 2 likewise comprises calculation means 60 which are connected to the measurement means and which, on the basis of the signal measured during the compression phase, are capable of emitting a negative diagnostic signal when the arterial pulse in the portion to be examined is higher than a diagnostic threshold value, and of emitting a positive diagnostic signal when this arterial pulse is lower than this diagnostic threshold value.

More precisely, these calculation means 60 are capable of deducing, from the signal measured during the compression phase, a useful signal that is characteristic of the arterial pulse in the portion to be examined 10b of the limb 10 of the patient. To do this, the measurement signal, which is composed of a pseudo-continuous spurious component and of a useful component having a measurement tension that is characteristic of the arterial pulse, is analyzed by the calculation means 60. These calculation means 60 comprise a high-pass filter with a cutoff frequency equal to 1 Hz, in such a way as to destroy the pseudo-continuous spurious component of the measurement signals, and also an operational amplifier capable of amplifying the measurement tension of the useful component.

The calculation means 60 are capable of comparing the amplified measurement tension with a predetermined comparison threshold tension, in such a way as to obtain a comparison tension having:
either a state “1” corresponding to a negative diagnostic signal when the amplitude of the amplified measurement tension is higher than the amplitude of the comparison threshold tension;
or a state “0” corresponding to a positive diagnostic signal when the amplitude of the amplified measurement tension is lower than the comparison threshold tension.

These diagnostic signals can then be transmitted to expression means 70 corresponding, for example, to a sound-emitting source. Thus, when the calculation means emit a negative diagnostic signal, the expression means 70 emit an acoustic signal, whereas, when the calculation means 60 emit a positive diagnostic signal, the expression means 70 do not emit an acoustic signal. According to this embodiment, the negative diagnostic information, corresponding to the absence of a defect, is a periodic emission of an acoustic signal, whereas the positive diagnostic information, corresponding to the presence of a defect, is the absence of an emission of an acoustic signal. Of course, other expression means 70 involving light, vibration or the like could also be envisioned.

The embodiments in FIGS. 7, 8a and 8b will now be described in detail.

FIGS. 7, 8a and 8b illustrate a second embodiment of a screening tool 102 according to the invention, in which the compression body 120 is formed by an inflatable sleeve 122 made of a flexible and atraumatic material, such as silicone.

This inflatable sleeve 122 has a first end, a second end, and an internal face, called the contact surface 124, intended to bear on the portion to be compressed 10a of the limb 10 of the patient. The inflatable sleeve 122 comprises fastening means 126 arranged on its two ends, allowing these to be kept fastened to each other. These fastening means 126 correspond, for example, to two complementary portions of Velcro® material. The inflatable sleeve 122 has a length sufficient to surround the portion to be compressed 10a of the limb 10 of the patient, and a width greater than 2 millimeters, preferably equal to 25 millimeters.

In this way, when the inflatable sleeve 122 engages with the portion to be compressed 10a, the contact surface 124 comes into non-punctiform or virtually non-punctiform contact with this portion to be compressed 10a, without causing it trauma, but in such a way as to compress it substantially homogeneously, without causing the patient discomfort.

The inflatable sleeve 122 comprises an outer and peripheral wall 122a, a fluid inlet 122b corresponding to a structural and functional opening made in the outer and peripheral wall 122a, and an internal chamber 122c delimited by the outer and peripheral wall and capable of containing, in a hermetic manner, a fluid introduced into this internal chamber 122c by way of the fluid inlet 122b.

According to this second embodiment, the screening tool 102 likewise comprises compression means 130 capable of compressing the contact surface 124 of the inflatable sleeve 122 against the portion to be compressed 10a. These compression means 130 comprise a delivery member 132 capable of generating a flow of fluid, and transfer means 134 capable of transporting the fluid from the delivery member 132 as far as the fluid inlet 122b of the compression body 120, in such a way as to transfer the fluid from the compression means 130 to the internal chamber 122c of the inflatable sleeve 122 via this fluid inlet 122b. More particularly, the delivery member 132 shown in FIG. 6 is composed of a bellows (or pear). Alternatively, however, this delivery member 132 could likewise be composed of any other analogous system, such as pump, allowing the internal chamber 122c of the inflatable sleeve 122 to be rapidly inflated.

According to one embodiment, the inflatable sleeve 122 used can be disposable and can be replaced for each new screening operation. It is thus possible to use the compression means 130 several times but to systematically replace the inflatable sleeve 122. To do this, it is necessary, at each screening operation, to connect the transfer means 134 to a new disposable inflatable sleeve 122, and the risks of bacterial contamination between the patients are thus limited.

The compression means 130 additionally comprise a calibrated valve 136 capable of allowing some of the fluid to escape from the internal chamber 122c of the inflatable sleeve 122 as long as said fluid generates, in this internal chamber 122c, a measurement pressure higher than a predetermined pressure threshold value.

Thus, in order to use the compression means 130, it is necessary firstly to place the inflatable sleeve 122 tightly around the portion to be compressed 10a and fasten the first and second ends by way of the fastening means 126.

By convention, this position is called the relaxed position, since the inflatable sleeve 122 is deflated.

Thereafter, it is necessary to actuate, either manually or automatically, the delivery member 132 in such a way as to transfer fluid, often air, to the internal chamber 122c in order to inflate the latter, and to do this until the predetermined pressure threshold value is reached or exceeded. When this pressure threshold value is exceeded, the calibrated valve 136 is responsible for evacuating some of this fluid, in order to reach substantially the predetermined pressure threshold value, and does so during a compression phase.

By convention, when the inflatable sleeve 122 is inflated, this is referred to as the tensioned position.

In the tensioned position, the contact surface generates, on said portion to be compressed 10a, a measurement pressure substantially equal to a previously determined pressure threshold value, and it does this throughout the measurement phase. By virtue of the calibrated valve 136, it is additionally possible to regulate the desired pressure threshold value precisely, simply, and without risk of subsequent loss of control. This pressure threshold value can be between 30 and 80 mmHg (millimeters of mercury) depending on the screening operations to be performed.

It is also possible to allow the measurement pressure to vary, within limited proportions, around this pressure threshold value during the measurement phase. This could be done, for example, by initiating the measurement phase before the calibrated valve 136 has finished extracting the remaining amount of fluid for obtaining, in the internal chamber 122c, a measurement pressure equal to the pressure threshold value.

Moreover, according to one particular embodiment, the calibrated valve 136 is capable of changing from a first state, in which the pressure threshold value corresponds to a first predetermined threshold value, to a second state, in which the pressure threshold value corresponds to a second predetermined threshold value. In this way, the screening tool 102 can easily perform two diagnoses under different measurement conditions, without incurring additional costs.

In addition, according to particular embodiments, the compression means can be obtained by any other suitable technique. More particularly, rather than introducing a fluid into the internal chamber 122c of the inflatable sleeve 122, the compression means could involve aspirating a fluid from this internal chamber 122c of the inflatable sleeve 122. Depending on the geometry of the inflatable sleeve 122, such aspiration could make it possible to generate a vacuum in the internal chamber 122c and thereby deform the inflatable sleeve 122 in such a way as to compress the portion to be compressed 10a of the limb 10 of the patient.

According to the embodiment in FIGS. 7 and 8a, the second embodiment of the screening tool 102 likewise comprises measurement means 140 capable of engaging structurally and functionally with the portion to be examined 10b of the limb 10 of the patient. These measurement means 140 likewise comprise a light source 150, such as a laser diode, a receiver 152, such as a photodiode, and a calculation unit 154. The light source 150 is capable of emitting rays of light toward the portion to be examined 10b of the limb 10 of the patient, these emitted rays of light having predetermined emission characteristics. The receiver 152, for its part, is capable of receiving the rays of light backscattered by the portion to be examined 10b of the limb 10 of the patient, these received rays of light likewise having predetermined reception characteristics.

Such measurement means 140 thus make it possible to carry out a measurement by backscattering during a phase referred to as the measurement phase.

According to the embodiment in FIG. 8a, the measurement means 140 comprise a receiving element 156, in this case a protective shell, having an outer protective wall 156a that delimits a protected inner space 156b. The light source 150 and the receiver 152 are positioned at least partially inside the protected inner space 156b. This receiving element 156 is arranged against the contact surface 124 of the inflatable sleeve 122 and can alternatively be fixed permanently on this inflatable sleeve 122, which ensures optimal positioning, or can be detached from this inflatable sleeve and fitted in position by the user, which allows it to be arranged more freely, especially in the case of a disposable inflatable sleeve 122.

According to an alternative embodiment shown in FIG. 8b, the measurement means 140 likewise comprise a receiving element 156 having an outer protective wall 156a that delimits a protected inner space 156b in which the light source 150 and the receiver 152 are arranged. However, according to this embodiment, the receiving element 156 is not arranged against the contact surface 124 of the inflatable sleeve 122 but against the inner face of the outer and peripheral wall 122a, in the internal chamber 122c of this inflatable sleeve 122a. In this case, the information transmitted by the measurement means 140 can be so transmitted by a radio transmission unit 158.

It should be noted that, in the embodiments in FIGS. 8a and 8b, the compression body 130 and the measurement means 140 are arranged in such a way that the portion to be compressed 10a corresponds at least partially to the portion to be examined 10b. However, this could not be the case for the example in FIG. 8a.

Moreover, in the second embodiment illustrated in FIGS. 7, 8a and 8b, the one calculation unit 154 is electrically connected to the light source 150 and to the receiver 152 and is likewise situated inside the protected inner space 156b or, according to one variant, outside this protected space 156b. In the same way as before, this calculation unit 154 makes it possible to compare the emission characteristics of the emitted rays of light with the reception characteristics of the received rays of light. Thus, by optical detection of one or more components of the blood, this calculation unit 154 can generate a measured signal corresponding to the measurement signal generated by the measurement means 140 during the compression phase. The measured signal is thus characteristic of the arterial pulse in the portion to be examined 10b when the latter is subjected to the measurement pressure. This embodiment is advantageous since, given that the measured signal is generated during the compression phase, it is not necessary to correlate it with the development of the pressure.

It should be emphasized that, according to this embodiment, the compression body 120 and the compression means 130 are functionally independent of the measurement means 140. Indeed, there is no electrical connection linking this compression body 120 and these compression means 130 to the measurement means 140. To use the screening tool 102, it is therefore necessary, firstly, to compress the portion to be compressed 10a by way of the compression body 120 and the compression means 130, then activate the measurement means 140 and hold the compression means 130 for a sufficient length of time such that the signal measured during this compression phase allows the calculation means 160 to generate a diagnostic signal. A preliminary measurement can additionally be carried out, before compression, in order to ascertain that the mechanism is functioning properly. However, according to an alternative embodiment, provision can be made to connect the compression means 130 to the measurement means 140 so as to transmit an activation signal to these measurement means 140 when the pressure threshold value is reached. Thus, the measurement means 140 can automatically activate as soon as, and for as long as, the portion to be compressed 10a is compressed as it should be.

The screening tool 102 likewise comprises calculation means 160 connected to the measurement means 140 by any suitable wired or wireless transmission element. On the basis of the signal measured during the compression phase, these calculation means 160 are capable of emitting a negative diagnostic signal when the arterial pulse in the portion to be examined is higher than a diagnostic threshold value, and of emitting a positive diagnostic signal when this arterial pulse is lower than this diagnostic threshold value.

More precisely, these calculation means 160 are capable of deducing, from the signal measured during the compression phase, a useful signal that is characteristic of the arterial pulse in the portion to be examined of the limb of the patient. To do this, the measurement signal, which is composed of a pseudo-continuous spurious component and of a useful component having a measurement tension that is characteristic of the arterial pulse, is analyzed by the calculation means 160. These calculation means 160 comprise a high-pass filter with a cutoff frequency equal to 1 Hz capable of eliminating the pseudo-continuous spurious component of the measurement signals, and also an operational amplifier capable of amplifying the measurement tension of the useful component.

As before, said calculation means 160 then compare the amplified measurement tension with a predetermined comparison threshold tension, in such a way as to obtain a comparison tension having:
either a state “1” when the amplitude of the amplified measurement tension is higher than the amplitude of the comparison threshold tension;
or a state “0” when the amplitude of the amplified measurement tension is lower than the comparison threshold tension.

These diagnostic signals are then subjected to a supplementary analysis by the calculation means 160 in order to determine, within a predetermined time interval, the number of negative diagnostic signals, corresponding to state “1”, that have been received. If this number is greater than a standard pulsation number, the calculation means emit a negative diagnostic signal corresponding to the absence of identification of a defect. Conversely, if this number is below a standard pulsation number, the calculation means emit a positive diagnostic signal corresponding to an identification of a defect.

Said diagnostic signal is then transmitted to expression means 170 composed of a light diode colored green 170a and a light diode colored red 170b. If a negative diagnostic signal is received, the expression means 170 activate the diode colored green 170a. If a positive diagnostic signal is received, the expression means 170 activate the diode colored red 170b. Such expression means 170 have the advantage of being better understood by a non-specialized user. However, other expression means 70 using light, vibrations or similar could also be envisioned.

The embodiment in FIGS. 8 and 9 will now be described in detail.

FIGS. 8 and 9 illustrate a third embodiment of a screening tool according to the invention, which is similar to the second embodiment set out above, but in which the measurement means function by transmission, not by backscatter.

As in the second embodiment, the measurement means 140 are capable of engaging structurally and functionally with the portion to be examined 10b of the limb 10 of the patient. These measurement means 140 comprise a light source 150, such as a laser diode, a receiver 152, such as a photodiode, and a calculation unit 154. The light source 150 is capable of emitting rays of light toward the portion to be examined 10b of the limb 10 of the patient, these emitted rays of light having predetermined emission characteristics. However, in contrast to the second embodiment, the receiver 152 of this third embodiment is, for its part, capable of receiving the rays of light having passed through the portion to be examined 10b of the limb 10 of the patient.

Thus, the receiver 152 is arranged in a separate receiving element 156 that is structurally independent of the receiving element 156 comprising the light source 150. In addition, these receiving elements 156 are arranged inside the inflatable sleeve 122, or optionally outside this inflatable sleeve 122, such that, in the position of use, the light source 150 emits rays of light in the direction of the receiver 152. In addition, with the light source 150 and the receiver 152 being separated from each other, each of the receiving elements can likewise comprise a calculation unit 154 and a communication unit 155 allowing the light source 150 and the receiver 152 to communicate with each other and/or with the calculation means 160.

It should be emphasized that, according to a particular configuration, one or other of the first, second and third embodiments described above can comprise heating means that are capable of maintaining or increasing the temperature of at least one portion of the limb of the patient. These heating means may thus make it possible to maintain or increase the temperature of the whole limb 10 of the patient, or simply one and/or the other of the portions to be compressed 10a and to be examined 10b. These heating means can likewise comprise a temperature probe capable of measuring the temperature in proximity to the portion to be covered of the patient.

These heating means can be provided in the form of a simple item of clothing that may or may not be disposable, for example a sock covering the whole of the foot, or a bag containing a heat-exchange fluid capable of increasing in temperature under the effect of a thermal activation signal, or else any other equivalent means.

In the case where the heating means correspond to an item of clothing, it should be noted that the latter can be formed from a thermally insulating fiber, in such a way as to efficiently maintain or increase the temperature of the one or more covered portions of the limb 10 of the patient.

In addition, said item of clothing may be capable of insulating the big toe from the other toes of the patient and can incorporate the temperature probe, the compression body 20, 120, the compression means 30, 130 and/or the measurement means 40, 140.

The invention is not limited to the examples described and shown in the text and in the attached drawings, and instead it extends to any other configuration that is feasible in light of the general understanding of a person skilled in the art.

For example, the invention likewise extends to an embodiment using, on the one hand, the measurement means 40 described with reference to the first embodiment illustrated by FIGS. 1 and 5 and, on the other hand, an inflatable sleeve 122 such as the one described with reference to the second and third embodiments illustrated by FIGS. 7 to 10. Said inflatable sleeve 122 could then be permanently fixed, directly or indirectly, on the inner face of the peripheral wall 42 and constitute a part or all of the contact surface.

1-39. (canceled)

40. A screening tool (2, 102) intended to identify a defect such as peripheral arterial disease of the lower limbs in a patient, comprising:
a compression body (20, 120) having a contact surface (24, 124), the compression body (20, 120) being capable of engaging with a portion to be compressed (10a) of a limb (10) of the patient, such that the contact surface (24, 124) comes into non-punctiform or virtually non-punctiform contact with the portion to be compressed (10a) of the limb (10) of the patient;
compression means (30, 130) capable of compressing this contact surface (24, 124) against this portion to be compressed (10a) of the limb (10) of the patient, in such a way as to generate, on said portion to be compressed (10a), a measurement pressure that is substantially homogeneous and equal to a pressure threshold value during a compression phase;
measurement means (40, 140) capable, firstly, of engaging structurally and functionally with a portion to be examined (10b) of the limb (10) of the patient and, secondly, of generating, by optical detection of one or more components of the blood, a measurement signal that is characteristic of an arterial pulse in the portion to be examined (10b) of the limb (10) of the patient during a measurement phase;
wherein the screening tool (2, 102) also comprises calculation means (60, 160) which, on the basis of a measured signal corresponding to the measurement signal generated by the measurement means (40, 140) during the compression phase, are capable of emitting a negative diagnostic signal when the arterial pulse in the portion to be examined is higher than a diagnostic threshold value, and of emitting a positive diagnostic signal when this arterial pulse is lower than this diagnostic threshold value;
and the screening tool (2, 102) additionally comprises expression means (70, 170) capable of expressing negative diagnostic information upon receipt of the negative diagnostic signal from the calculation means (60, 160).

41. The screening tool (2, 102) as claimed in claim 40, in which the compression body (20, 120) comprises at least one flexible portion (46a, 46b, 122a) made of a flexible and atraumatic material allowing the compression body to be held in position, such as silicone.

42. The screening tool (2, 102) as claimed in claim 41, in which the compression body (20, 120) comprises the at least one flexible portion (46a, 46b, 122a) and a rigid or semi-rigid peripheral wall (42); in which the at least one flexible portion (46a, 46b, 122a) is permanently fixed, directly or indirectly, on the peripheral wall (42); and in which the contact surface (24, 124) is formed partly or wholly on the at least one flexible portion (46a, 46b, 122a).

43. The screening tool (102) as claimed in claim 42, in which the at least one flexible portion (122a) comprises an internal chamber (122c) capable of containing, in a hermetic manner, a fluid introduced into this internal chamber (122c) by way of a fluid inlet (122b).

44. The screening tool (102) as claimed in claim 40, in which the compression body (120) comprises a sleeve (122), with:
an outer and peripheral wall (122a) made of a flexible and atraumatic material;
a fluid inlet (122b) corresponding to a structural and functional opening made in the outer and peripheral wall (122a); and
an internal chamber (122c) delimited by the outer and peripheral wall (122a) and capable of containing, in a hermetic manner, a fluid introduced into this internal chamber (122c) by way of the fluid inlet.

45. The screening tool (102) as claimed in claim 43, in which the compression means (130) comprise a delivery member (132) capable of generating a flow of fluid, and transfer means (134) capable of transporting the fluid from the delivery member (132) as far as the fluid inlet (122b) of the compression body (120), in such a way as to transfer the fluid from the compression means (130) to the internal chamber (122c) of the compression body (120) via this fluid inlet (122b).

46. The screening tool (2) as claimed in claim 40, in which the compression body (20) comprises an inextensible band (22) capable of being deformed in such a way as to fit tightly on the portion to be compressed (10a) of the limb (10) of the patient; the band having a width of greater than 2 mm (millimeters).

47. The screening tool (2, 102) as claimed in claim 40, in which the measurement means (40, 140) comprise:
a light source (50, 150), such as a laser diode, capable of emitting rays of light toward the portion to be examined (10b) of the limb (10) of the patient, the emitted rays of light having predetermined emission characteristics;
a receiver (52, 152), such as a photodiode, capable of receiving the rays of light transmitted or backscattered by the portion to be examined (10b) of the limb (10) of the patient, the received rays of light having predetermined reception characteristics;
a calculation unit (54, 154) capable of comparing the emission characteristics with the reception characteristics, in such a way as to deduce therefrom the measurement signal that is characteristic of the arterial pulse in the portion to be examined of the limb (10) of the patient.

48. The screening tool (2, 102) as claimed in claim 47, in which the calculation means (54, 154) are capable of deducing, from the measurement signal, a useful signal that is characteristic of the arterial pulse in the portion to be examined (10b) of the limb (10) of the patient.

49. The screening tool (2, 102) as claimed in claim 48, in which, when the measurement signal is composed of a pseudo-continuous spurious component and of a useful component having a measurement tension that is characteristic of the arterial pulse, the calculation means (54, 154) are capable of eliminating the pseudo-continuous spurious component of the measurement signals and of amplifying the measurement tension of the useful component.

50. The screening tool (2, 102) as claimed in claim 49, in which the calculation means (54, 154) are capable of comparing the amplified measurement tension with a comparison threshold tension, in such a way as to emit a negative diagnostic signal when the amplitude of the amplified measurement tension is higher than the amplitude of the comparison threshold tension, and a positive diagnostic signal when the amplitude of the amplified measurement tension is lower than the comparison threshold tension.

51. The screening tool (2, 102) as claimed in claim 47, in which the light source (50, 150) and the receiver (52, 152) are capable of being positioned on either side of the portion to be examined (10b) of the limb (10) of the patient, such that the received rays of light correspond to the emitted rays of light having passed through the portion to be examined (10b) of the limb (10) of the patient or the light source (50, 150) is capable of being positioned in proximity to the receiver (52, 152), such that the received rays of light correspond to the emitted rays of light backscattered by the portion to be examined (10b) of the limb (10) of the patient.

52. The screening tool (2, 102) as claimed in claim 40, comprising heating means that are capable of maintaining or increasing the temperature of at least one portion of the limb (10) of the patient.

53. The screening tool (2, 102) as claimed in claim 40, wherein the measurement means (40, 140) are capable of engaging structurally and functionally with a portion to be examined (10b) of the limb (10) of the patient and of generating, by optical detection of one or more components of the blood, a measurement signal that is characteristic of an arterial pulse in the portion to be examined (10b) of the limb (10) of the patient during a measurement phase without variable compression of the arteries.

54. The screening tool (2, 102) as claimed in claim 40, in which the compression means (20, 120) are capable of generating, on the portion to be compressed (10a), a fixed measurement pressure situated in a range of 30 to 80 mmHg (millimeters of mercury).
